(19) United States
(12) Patent Application Publication
(10) Pub. No.: US 2026/0041560 A1
(43) Pub. Date: Feb. 12, 2026

(54) TIBIAL PROSTHESIS SYSTEM

(71) Applicant: Optimotion Implants LLC, Orlando, FL (US)

(72) Inventors: Vuong Binh NGUYEN, Windermere, FL (US); Andrew RYNEARSON, Winter Springs, FL (US)

(21) Appl. No.: 18/800,028

(22) Filed: Aug. 10, 2024

(51) Int. Cl. A61F 2/38; A61F 2/30

(52) U.S. Cl. CPC A61F 2/389; A61F 2002/30331

(57) ABSTRACT

A tibial prosthesis system for knee arthroscopy may include a tibial tray having an anterior portion, a posterior portion, a bone facing surface, a joint facing surface, and a central aperture. The tibial prosthesis system may also include a clip configured to be slidably received in the central aperture. The posterior portion may include a posterior notch. The central aperture may extend posteriorly from the anterior portion, and from the bone facing surface to the joint facing surface. At least a portion of the joint facing surface may extend between the central aperture and the posterior notch.

### TECHNICAL FIELD

The present disclosure relates to systems and methods for use in orthopedic surgery. More specifically, the present disclosure relates to tibial prosthesis systems used in knee arthroplasty.

### BACKGROUND

Knee arthroplasty is a common procedure which involves replacing a part or whole of a patient's knee joint and replacing it with an implant. This surgery is often undertaken in order to reduce pain and increase mobility in patients with damaged and/or diseased knee joints. During the surgery, the damaged joint is replaced with a prosthetic implant, which typically includes a femoral component, a tibial component (tibial tray), and a polyethylene insert. The tibial tray is a critical part of the knee implant system, providing a stable platform for the polyethylene insert and the articulation of the knee joint.

Despite the high success rate of knee arthroplasty, one of the challenges encountered is the management of the anterior cruciate ligament (ACL). The ACL is a crucial stabilizing ligament of the knee, and its preservation or repair during arthroplasty can significantly influence post-operative stability and patient outcomes. Recent advancements in knee arthroplasty have focused on the development of implants that allow for the preservation of the ACL. However, there is a notable lack of solutions that provide specific features for the repair or augmentation of the ACL post-arthroplasty. Addressing this gap can enhance the functional outcomes of knee replacements by maintaining or restoring the natural biomechanics of the knee joint.

There exists a need for an implant which can be used in knee arthroplasty procedures without creating needless complications in ACL repair procedures.

### SUMMARY

The various systems and methods of the present disclosure have been developed in response to the present state of the art, and in particular, in response to the problems and needs in the art that have not yet been fully solved by currently available tibial prosthesis systems used in knee arthroplasty.

In some embodiments, a tibial prosthesis system for knee arthroscopy may include a tibial tray having an anterior portion, a posterior portion, a bone facing surface, a joint facing surface, and a central aperture. The tibial prosthesis system may also include a clip configured to be slidably received in the central aperture. The posterior portion may include a posterior notch. The central aperture may extend posteriorly from the anterior portion, and from the bone facing surface to the joint facing surface. At least a portion of the joint facing surface may extend between the central aperture and the posterior notch.

In the tibial prosthesis system of any preceding paragraph, the central aperture may be configured to receive a natural ligament or a ligament graft.

In the tibial prosthesis system of any preceding paragraph, the tibial tray may include a central axis equidistant between the anterior portion and the posterior portion, and the central aperture may extend posteriorly beyond the central axis.

In the tibial prosthesis system of any preceding paragraph, the clip may include an anterior profile configured to align with an anterior edge of the anterior portion of the tibial tray.

In the tibial prosthesis system of any preceding paragraph, the clip may extend from the bone facing surface to the joint facing surface.

In the tibial prosthesis system of any preceding paragraph, with the clip received in the central aperture, at least a portion of the central aperture may remain unobstructed between the bone facing surface and the joint facing surface.

In the tibial prosthesis system of any preceding paragraph, with the clip received in the central aperture, the central aperture may be completely obstructed.

In the tibial prosthesis system of any preceding paragraph, the system may further include a first stem and a second stem, wherein both the first stem and the second stem may be configured to be embedded within a tibia to inhibit movement of the tibial prosthesis system relative to the tibia.

In some embodiments, a tibial prosthesis system for knee arthroscopy may include a tibial tray having an anterior portion, a posterior portion, a central axis, and a central aperture. The tibial prosthesis system may also include a clip configured to be received in the central aperture. The central axis may be equidistant between the anterior portion and the posterior portion. The posterior portion may include a posterior notch. The central aperture may extend posteriorly from the anterior portion beyond the central axis.

In the tibial prosthesis system of any preceding paragraph, the central aperture may be configured to receive a natural ligament or a ligament graft.

In the tibial prosthesis system of any preceding paragraph, with the clip received in the central aperture, at least a portion of the central aperture may remain unobstructed.

In the tibial prosthesis system of any preceding paragraph, with the clip received in the central aperture, the central aperture may be completely obstructed.

In the tibial prosthesis system of any preceding paragraph, the tibial prosthesis system may further include a first stem and a second stem, wherein both the first stem and the second stem may be configured to be embedded within a tibia to inhibit movement of the tibial prosthesis system relative to the tibia.

In the tibial prosthesis system of any preceding paragraph, the clip may include an anterior profile configured to align with an anterior edge of the anterior portion of the tibial tray.

In the tibial prosthesis system of any preceding paragraph, the tibial tray may further include a bone facing surface and a joint facing surface. The clip may extend from the bone facing surface to the joint facing surface.

In some embodiments, a tibial prosthesis system for knee arthroscopy may include an anterior portion, a bone facing surface, a joint facing surface, and a central aperture. The tibial prosthesis system may also include a clip configured to be received in the central aperture. The central aperture may extend posteriorly from the anterior portion, and from the bone facing surface to the joint facing surface. The central aperture may be configured to receive a natural ligament or a ligament graft.

In the tibial prosthesis system of any preceding paragraph, the tibial tray may further include a posterior portion and a central axis equidistant between the anterior portion and the posterior portion, and the central aperture may extend posteriorly beyond the central axis.

In the tibial prosthesis system of any preceding paragraph, with the clip received in the central aperture, at least a portion of the central aperture may remain unobstructed between the bone facing surface and the joint facing surface.

In the tibial prosthesis system of any preceding paragraph, the tibial prosthesis system may further include a first stem and a second stem, wherein both the first stem and the second stem may be configured to be embedded within a tibia to inhibit movement of the tibial prosthesis system relative to the tibia.

In the tibial prosthesis system of any preceding paragraph, the clip may include an anterior profile configured to align with an anterior edge of the anterior portion of the tibial tray.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the present disclosure will become more fully apparent from the following description taken in conjunction with the accompanying drawings. Understanding that these drawings depict only exemplary embodiments and are, therefore, not to be considered limiting of the scope of the present disclosure, the exemplary embodiments of the present disclosure will be described with additional specificity and detail through use of the accompanying drawings.

FIG. 1A is a perspective view of a tibial prosthesis according to an embodiment of the present disclosure.

FIG. 1B is a top view of the tibial prosthesis of FIG. 1A.

FIG. 1C is a side view of the tibial prosthesis of FIG. 1A.

FIG. 1D is a front view of the tibial prosthesis of FIG. 1A.

FIG. 2A is a perspective view of the tibial prosthesis of FIG. 1A.

FIG. 2B is an exploded perspective view of the tibial prosthesis of FIG. 1A.

FIG. 3A is a perspective view of a tibial tray according to an embodiment of the present disclosure.

FIG. 3B is a top view of the tibial tray of FIG. 3A.

FIG. 3C is a side view of the tibial tray of FIG. 3A.

FIG. 3D is a front view of the tibial tray of FIG. 3A.

FIG. 4A is a perspective view of a clip according to an embodiment of the present disclosure.

FIG. 4B is a top view of the clip of FIG. 4A.

FIG. 4C is a side view of the clip of FIG. 4A.

FIG. 4D is a front view of the clip of FIG. 4A.

FIG. 5 is a perspective view of a tibial prosthesis according to an embodiment of the present disclosure.

It is to be understood that the drawings are for purposes of illustrating the concepts of the present disclosure and may not be drawn to scale. Furthermore, the drawings illustrate exemplary embodiments and do not represent limitations to the scope of the present disclosure.

### DETAILED DESCRIPTION

Exemplary embodiments of the present disclosure will be best understood by reference to the drawings, wherein like parts are designated by like numerals throughout. It will be readily understood that the components of the present disclosure, as generally described and illustrated in the drawings, could be arranged, and designed in a wide variety of different configurations. Thus, the following more detailed description of the embodiments of the devices, systems, and methods, as represented in the drawings, is not intended to limit the scope of the present disclosure but is merely representative of exemplary embodiments of the present disclosure.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in the drawings, the drawings are not necessarily drawn to scale unless specifically indicated.

Standard medical planes of reference and descriptive terminology are employed in this specification. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body. Proximal means toward the trunk of the body. Proximal may also mean toward a user or operator. Distal means away from the trunk. Distal may also mean away from a user or operator. Dorsal means toward the top of the foot. Plantar means toward the sole of the foot. Varus means deviation of the distal part of the leg below the knee inward, resulting in a bowlegged appearance. Valgus means deviation of the distal part of the leg below the knee outward, resulting in a knock-kneed appearance.

The present disclosure relates to tibial prosthesis systems and methods used in knee arthroplasty. Those skilled in the art will recognize that the following description is merely illustrative of the principles of the technology, which may be applied in various ways to provide many alternative embodiments. It will be understood that other variations and uses are contemplated, including, but not limited to, applications in total knee arthroplasty (TKA) and/or unicompartmental (partial) knee arthroplasty (UKA).

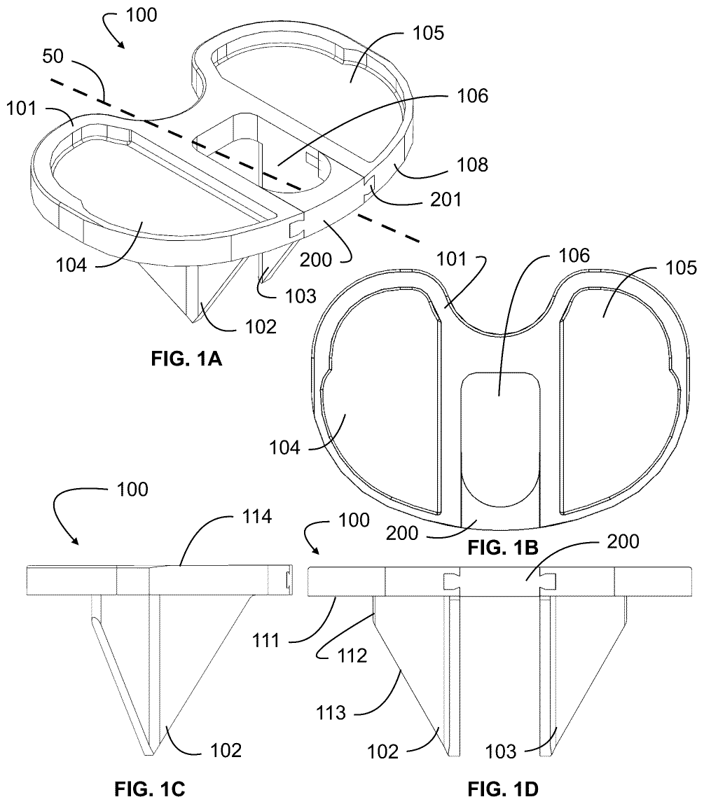

FIG. 1A is a perspective view of a tibial prosthesis 100 according to an embodiment of the present disclosure. FIG. 1B is a top view of the tibial prosthesis 100, FIG. 1C is a side view of the tibial prosthesis 100, and FIG. 1D is a front view of the tibial prosthesis 100. The tibial prosthesis 100 may be configured so that, with the tibial prosthesis 100 implanted on a tibia, the tibial prosthesis 100 may not inhibit and/or interfere with an anterior cruciate ligament (ACL) repair and/or ACL reconstruction surgical procedure. The tibial prosthesis 100 may further be configured to receive a full tibial insert as part of a total knee arthroplasty (TKA) and/or a partial tibial insert as part of a unicompartmental (partial) knee arthroplasty (UKA).

Alternatively, the tibial prosthesis 100 may be configured to receive two partial tibial inserts including a medial tibial insert and a lateral tibial insert. The tibial prosthesis 100 may be configured so that an ACL reconstruction may be performed during a TKA or UKA procedure. Alternatively, the tibial prosthesis 100 may be configured so that an ACL reconstruction may be performed subsequent to a TKA and/or UKA procedure.

The tibial prosthesis 100 may include a tibial tray 101 and a clip 200. The tibial tray 101 may include a first stem 102, a second stem 103, a first tray portion 104, a second tray portion 105, and a central aperture 106. The first stem 102 and the second stem 103 may be configured to be embedded within a tibia to inhibit movement of the tibial prosthesis 100 relative to the tibia. The first stem 102 and the second stem 103 may be further configured to increase stability of a prosthetic knee joint by reducing micromotion of the tibial tray relative to the tibia.

The first stem 102 and/or the second stem 103 may have a tapered portion 113 that may decrease in cross-sectional area as the first stem 102 and/or second stem 103 extend away from a bone facing surface 111 of the tibial tray 101. Additionally, or alternatively, the first stem 102 and/or the second stem 103 may be configured as a pyramid, a cone, a trapezoidal prism, and/or other three-dimensional tapered form in which the stem decreases in cross-sectional area as the stem extends away from the bone facing surface 111.

Additionally, or alternatively, the first stem 102 and/or the second stem 103 may include one or more ribs that may decrease in cross-sectional area as the one or more ribs extend away from the bone facing surface 111. Additionally, or alternatively, the first stem 102 and/or the second stem 103 may include a straight portion 112 proximate the bone facing surface 111 and between the bone facing surface 111 and the tapered portion 113.

A joint facing surface 114 may include the first tray portion 104 and the second tray portion 105. The first tray portion 104 and/or the second tray portion 105 may each be configured to receive a partial tibial insert (not shown), for example, as is commonly used as a component of a UKA procedure. Alternatively, the first tray portion 104 and the second tray portion 105 may be configured to receive a single full tibial insert (not shown), for example, as is commonly used as a component of a TKA procedure. The full tibial insert may include an aperture that generally aligns with the central aperture 106.

The joint facing surface 114 may also include the central aperture 106 and a posterior notch 109. At least a portion of the joint facing surface 114 may extend between the central aperture 106 and the posterior notch 109. The central aperture 106 may extend posteriorly from an anterior portion 108 of the tibial tray 101. The central aperture 106 may be configured to receive a clip 200.

The central aperture 106 may be located within a central portion of the tibial tray 101 so that, with the tibial prosthesis 100 implanted on the tibia, a center of the central aperture 106 may be located generally within a middle third of where an ACL attaches to a tibial plateau. Alternatively, with the tibial prosthesis 100 implanted on the tibia, a center of the central aperture 106 may be located approximately 40% of the medial-to-lateral width of the interspinous distance, in line with a posterior edge of a lateral meniscus anterior horn, approximately 14mm-16mm anterior to a posterior cruciate ligament.

Alternatively, with the tibial prosthesis 100 implanted on the tibia, the central aperture 106 may be located approximately 1 cm-2 cm medial to a medial aspect of a tibial tubercle and 1 cm above an insertion point of a hamstring at a pes anserinus. Alternatively, with the tibial prosthesis 100 implanted on the tibia, the central aperture 106 may be located approximately 8 mm-10 mm posterior to an intermeniscal ligament, in line with an anterior horn of a lateral meniscus, and approximately 7 mm to 10 mm anterior to a posterior cruciate ligament insertion with the center of the central aperture 106 ideally being just posterior and medial to an anterior horn of a lateral meniscus.

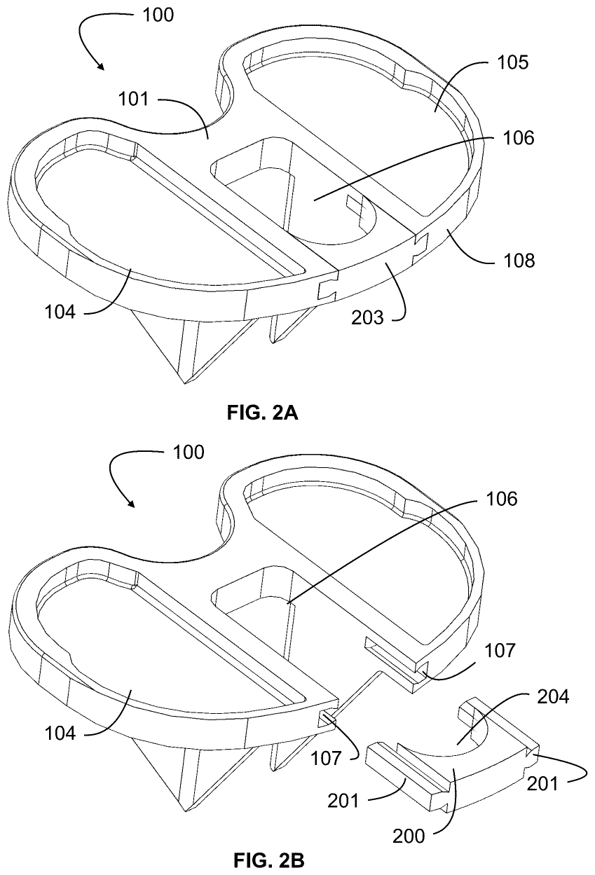

FIG. 2A is a perspective view of the tibial prosthesis 100. FIG. 2B is an exploded perspective view of the tibial prosthesis 100. The tibial prosthesis 100 may be configured to receive a clip 200. The clip 200 may be received in the anterior portion 108 of the tibial tray 101. The tibial prosthesis 100 may be configured to receive the clip 200 prior to implantation of the tibial prosthesis 100 on to the tibia. Alternatively, the tibial prosthesis 100 may be configured so that the tibial tray 101 may receive the clip 200 with the tibial tray 101 implanted on the tibia. The clip 200 may be received in the tibial tray 101 after an ACL, a natural ligament or a ligament graft 50, and/or other anatomic structures are received in the central aperture 106. The clip 200 may be received in the tibial tray 101 before or after one or both tibial inserts are received in the first tray portion 104 and/or the second tray portion 105.

The clip may include an anterior profile 203. The anterior profile 203 may be configured to align with an anterior edge 118 of the anterior portion 108 of the tibial tray 101 when the clip 200 is received within the tibial tray 101.

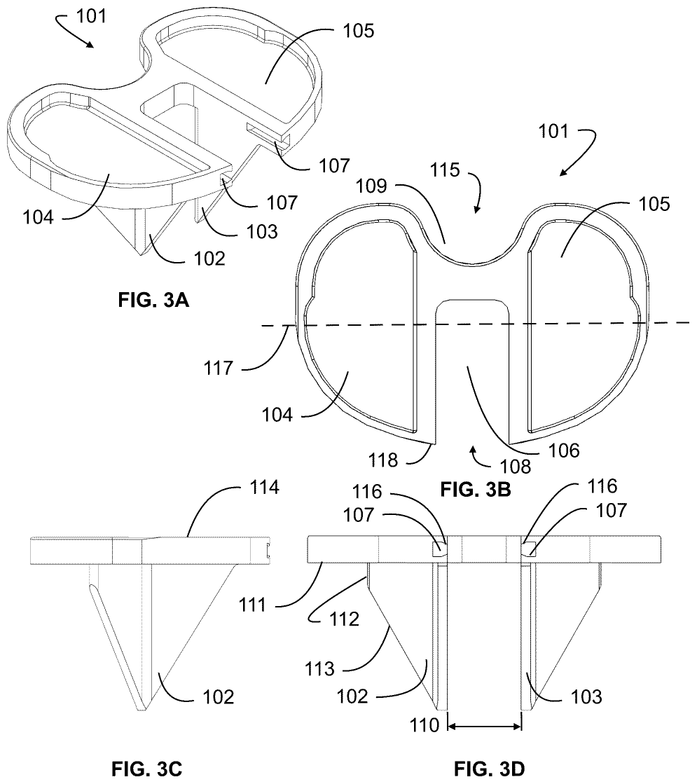

FIG. 3A is a perspective view of a tibial tray 101 according to an embodiment of the present disclosure. FIG. 3B is a top view of the tibial tray 101, FIG. 3C is a side view of the tibial tray 101, and FIG. 3D is a front view of the tibial tray 101. The tibial tray 101 may include a posterior portion 115, a posterior notch 109 and the central aperture 106. The central aperture 106 may include slots 107 and an aperture width 110.

The tibial tray 101 may also include a central axis 117 located equidistant from the anterior portion 108 and the posterior portion 115. The central aperture 106 may extend posteriorly from the anterior portion 108 beyond the central axis 117. The posterior notch 109 may extend anteriorly from the posterior portion 115 and may not extend beyond the central axis 117. The central aperture 106 may not intersect and/or overlap the posterior notch 109. With the tibial prosthesis 100 implanted on the tibia, the posterior notch 109 may not be configured to facilitate ACL repair and/or ACL reconstruction.

The central aperture 106 may include slots 107 configured to slidably receive the clip 200. The slots 107 may extend posteriorly from the anterior portion 108 so that, when the clip 200 is fully received within the slots 107, the anterior profile 203 of the clip 200 may be aligned with an anterior edge 118 of the anterior portion 108 of the tibial tray 101.

Alternatively, the central aperture 106 may include superior slots extending inferiorly from the joint facing surface. The superior slots may be configured to slidably receive the clip 200. Alternatively, the central aperture 106 may include a ledge portion configured to receive the clip 200. The ledge portion may include an undercut and/or a tab configured to engage a mating feature on the clip 200 so that, when the clip 200 is fully inserted into the ledge portion, the undercut and/or the tab may function as a locking feature to prevent the clip 200 from backing out of the ledge portion.

Additionally, or alternatively, the central aperture may include a groove configured to receive a snap ring type component configured to retain the clip 200 within the central aperture 106.

Alternatively, the anterior portion 108 may include one or more first apertures configured to receive one or more fasteners. Additionally, the clip 200 may include an extended portion having one or more second apertures aligned with the one or more first apertures of the anterior portion 108. The one or more fasteners may be received within the one or more first apertures and the one or more second apertures to secure the clip 200 to the anterior portion 108.

Additionally, or alternatively, the central aperture 106 may include an undercut and/or a tab configured to engage a mating feature on the clip 200 so that, when the clip 200 is fully inserted into the slots, the undercut and/or the tab may function as a locking feature to prevent the clip 200 from backing out of the slots.

The central aperture 106 may further include an aperture width 110. The aperture width 110 may be sized to facilitate an ACL repair and/or ACL reconstruction surgical procedure. The aperture width 110 may be large enough to allow passage of a drill, a reamer, a drill guide, a sizer, an obturator, a natural ligament or a ligament graft 50, a tendon graft, a bone-tendon-bone graft, a screw, and/or other instruments and/or implants used in an ACL repair and/or ACL reconstruction procedure.

Tunnel malposition may be a common technical reason for ACL reconstruction failure. Small variations in tibial tunnel placement may result in significant differences in patient outcome. Anatomic placement of a tibial tunnel may lead to greater knee stability and more accurate knee kinematics. The central aperture 106 may be located and sized to allow anatomic placement of tibial tunnels and/or femoral tunnels during an ACL reconstruction procedure.

The first stem 102 may be spaced apart from the second stem 103 by a distance that is equal to or greater than the aperture width 110. The aperture width 110 may be between 12 mm and 20 mm.

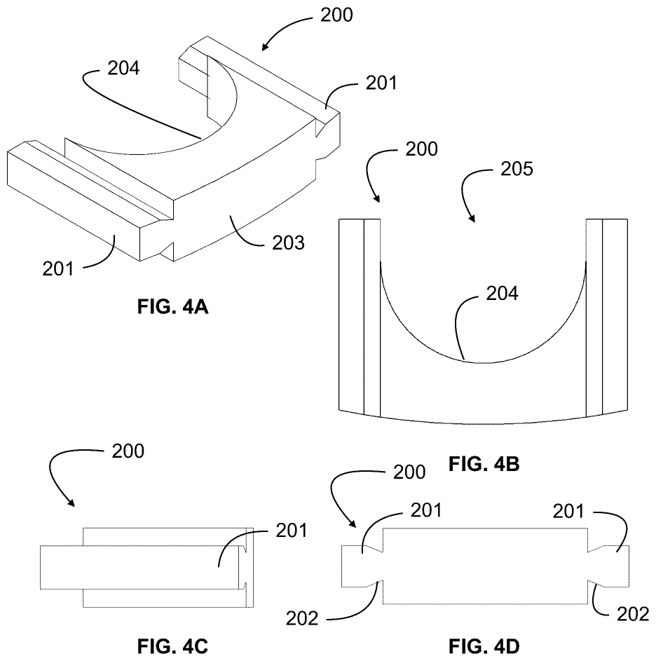

FIG. 4A is a perspective view of the clip 200 according to an embodiment of the present disclosure. FIG. 4B is a top view of the clip 200, FIG. 4C is a side view of the clip 200, and FIG. 4D is a front view of the clip 200. The clip 200 may be configured to be received within the central aperture 106 of the tibial tray 101. The clip 200 may further be configured to fill at least a portion of the central aperture 106.

The clip 200 may include rails 201, the anterior profile 203, and a posterior profile 204. The rails 201 may be configured to be slidably received with the slots 107 of the tibial tray 101. The rails 201 may include a tapered portion 202 corresponding to a tapered portion 116 in the slots 107. The tapered portion 202 and the tapered portion 116 may cooperate to inhibit distortion and/or expansion of the anterior portion 108 of the tibial tray 101. The tapered portion 202 and the tapered portion 116 may be configured as a mating dovetail feature.

Alternatively, the central aperture 106 may include rails and the clip 200 may include slots configured to receive the rails of the central aperture 106.

The posterior profile 204 may extend anteriorly from a posterior end 205 toward the anterior profile 203. The posterior profile 204 may have a generally semi-circular profile. Alternatively, the posterior profile 204 may have a generally rectangular profile which may include radiused corners. The posterior profile 204 may be configured so that, when the clip 200 is fully received within the tibial tray 101, at least a portion of the central aperture 106 remains unobstructed between the bone facing surface 111 and the joint facing surface 114.

Alternatively, the posterior profile 204 may generally align with the posterior end 205. The posterior profile 204 may have a generally straight profile. The posterior profile 204 may be configured so that, when the clip 200 is fully received within the tibial tray 101, at least a portion of the central aperture 106 remains unobstructed between the bone facing surface 111 and the joint facing surface 114.

Alternatively, the posterior profile 204 may extend posteriorly from the posterior end 205 away from the anterior profile 203. The posterior profile 204 may have a generally semi-circular profile. Alternatively, the posterior profile 204 may have a generally rectangular profile which may include radiused corners. The posterior profile 204 may be configured so that, when the clip 200 is fully received within the tibial tray 101, at least a portion of the central aperture 106 remains unobstructed between the bone facing surface 111 and the joint facing surface 114.

Alternatively, the posterior profile 204 may extend posteriorly from the posterior end 205 away from the anterior profile 203. The posterior profile 204 may have a profile generally matching the profile of the central aperture 106. The posterior profile 204 may be configured so that, when the clip 200 is fully received within the tibial tray 101, the central aperture 106 is completely obstructed between the bone facing surface 111 and the joint facing surface 114.

Alternatively, the posterior profile 204 may extend posteriorly from the posterior end 205 away from the anterior profile 203. The posterior profile 204 may include an aperture that may have an aperture width that is less than the aperture width 110 of the central aperture 106. The posterior profile 204 may be configured so that, when the clip 200 is fully received within the tibial tray 101, at least a portion of the central aperture 106 remains unobstructed between the bone facing surface 111 and the joint facing surface 114.

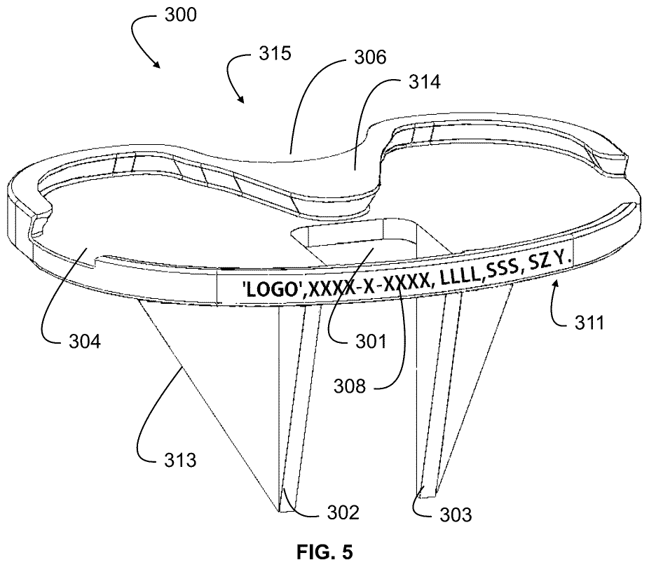

FIG. 5 is a perspective view of a tibial prosthesis 300 according to an embodiment of the present disclosure. The tibial prosthesis 300 may be configured so that, with the tibial prosthesis 300 implanted on a tibia, the tibial prosthesis 300 may not inhibit and/or interfere with an anterior cruciate ligament (ACL) repair and/or ACL reconstruction surgical procedure. The tibial prosthesis 300 may further be configured to receive a full tibial insert as part of a total knee arthroplasty (TKA) and/or a partial tibial insert as part of a unicompartmental (partial) knee arthroplasty (UKA).

Alternatively, the tibial prosthesis 300 may be configured to receive two partial tibial inserts including a medial tibial insert and a lateral tibial insert. The tibial prosthesis 300 may be configured so that an ACL reconstruction may be performed during a TKA or UKA procedure. Alternatively, the tibial prosthesis 300 may be configured so that an ACL reconstruction may be performed subsequent to a TKA and/or UKA procedure.

The tibial prosthesis 300 may include a first stem 302, a second stem 303, a tray portion 304, and a central aperture 301. The first stem 302 and the second stem 303 may be configured to be embedded within a tibia to inhibit movement of the tibial prosthesis 300 relative to the tibia. The first stem 302 and the second stem 303 may be further configured to increase stability of a prosthetic knee joint by reducing micromotion of the tibial prosthesis 300 relative to the tibia.

The first stem 302 and/or the second stem 303 may have a tapered portion 313 that may decrease in cross-sectional area as the first stem 302 and/or second stem 303 extend away from a bone facing surface 311 of the tibial prosthesis 300. Additionally, or alternatively, the first stem 302 and/or the second stem 303 may be configured as a pyramid, a cone, a trapezoidal prism, and/or other three-dimensional tapered form in which the stem decreases in cross-sectional area as the stem extends away from the bone facing surface 311.

Additionally, or alternatively, the first stem 302 and/or the second stem 303 may include one or more ribs that may decrease in cross-sectional area as the one or more ribs extend away from the bone facing surface 311.

A joint facing surface 314 may include the tray portion 304. The tray portion 304 may be configured to receive one or more partial tibial inserts (not shown), for example, as is commonly used as a component of a UKA procedure. Alternatively, the tray portion 304 may be configured to receive a full tibial insert (not shown), for example, as is commonly used as a component of a TKA procedure. The full tibial insert may include an aperture that generally aligns with the central aperture 301.

The joint facing surface 314 may also include the central aperture 301 and a posterior notch 306. The posterior notch 306 may extend anteriorly from a posterior portion 315. The central aperture 301 may not intersect and/or overlap the posterior notch 306. With the tibial prosthesis 300 implanted on the tibia, the posterior notch 306 may not be configured to facilitate ACL repair and/or ACL reconstruction. At least a portion of the joint facing surface 314 may extend between the central aperture 301 and the posterior notch 306.

The tibial prosthesis 300 may include an anterior portion 308. The central aperture 301 may be located between the posterior notch 306 and the anterior portion 308.

The central aperture 301 may be located within a central portion of the tibial prosthesis 300 so that, with the tibial prosthesis 300 implanted on the tibia, a center of the central aperture 301 may be located generally within a middle third of where an ACL attaches to a tibial plateau. Alternatively, with the tibial prosthesis 300 implanted on the tibia, a center of the central aperture 301 may be located approximately 40% of the medial-to-lateral width of the interspinous distance, in line with a posterior edge of a lateral meniscus anterior horn, approximately 14 mm-16 mm anterior to a posterior cruciate ligament.

Alternatively, with the tibial prosthesis 300 implanted on the tibia, the central aperture 301 may be located approximately 1 cm-2 cm medial to a medial aspect of a tibial tubercle and 1 cm above an insertion point of a hamstring at a pes anserinus. Alternatively, with the tibial prosthesis 300 implanted on the tibia, the central aperture 301 may be located approximately 8 mm-10 mm posterior to an intermeniscal ligament, in line with an anterior horn of a lateral meniscus, and approximately 7 mm to 10 mm anterior to a posterior cruciate ligament insertion with the center of the central aperture 301 ideally being just posterior and medial to an anterior horn of a lateral meniscus.

The central aperture 301 may be sized to facilitate an ACL repair and/or ACL reconstruction surgical procedure. The central aperture 301 may be large enough to allow passage of a drill, a reamer, a drill guide, a sizer, an obturator, a natural ligament or a ligament graft 50, a tendon graft, a bone-tendon-bone graft, a screw, and/or other instruments and/or implants used in an ACL repair and/or ACL reconstruction procedure. The central aperture 301 may be located and sized to allow anatomic placement of tibial tunnels and/or femoral tunnels during an ACL reconstruction procedure. The first stem 302 may be spaced apart from the second stem 303 by a distance that is equal to or greater than a width of the central aperture 301. The width of the central aperture 301 may be between 12 mm and 20 mm.

An exemplary method for performing an ACL reconstruction in conjunction with a knee arthroplasty may include the following steps:
1. Insert a clip into a tibial tray having a central aperture to form an assembled tibial prosthesis.
2. Implant the assembled tibial prosthesis onto a tibia adjacent to a knee joint.
3. Perform an ACL reconstruction in which a ligament graft is passed through the central aperture.

Those of skill in the art will recognize that this is only one of many potential methods that may be used for performing an ACL reconstruction in conjunction with a knee arthroplasty. In alternative embodiments, the ligament graft may be passed through the central aperture before the tibial prosthesis is implanted onto the tibia. Additionally, or alternatively, the ligament graft may be received in the central aperture before the clip is inserted into the tibial tray. Additionally, or alternatively, the ACL reconstruction and/or the ACL repair procedure may be performed subsequent to a TKA and/or a UKA procedure.

In alternative embodiments, different methods may be used for performing an ACL reconstruction in conjunction with a knee arthroplasty other than the methods described above. Further, the methods set forth above may be used for performing an ACL reconstruction in conjunction with a knee arthroplasty utilizing a tibial prosthesis besides those specifically disclosed herein.

An exemplary method for performing an ACL sparing knee arthroplasty may include the following steps:
1. Implant a tibial tray having a central aperture onto a tibia adjacent to a knee joint.
2. Guide an ACL into the central aperture of the tibial tray without cutting the ACL.
3. Insert a clip into the central aperture of the tibial tray without impinging the ACL.

Those of skill in the art will recognize that this is only one of many potential methods that may be used for performing an ACL sparing knee arthroplasty. In alternative embodiments, different methods may be used for performing an ACL sparing knee arthroplasty other than the methods described above. Further, the methods set forth above may be used for performing an ACL sparing knee arthroplasty utilizing a tibial prosthesis besides those specifically disclosed herein.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the present disclosure. This method of disclosure, however, is not to be interpreted as reflecting an intention that any embodiment requires more features than those expressly recited in that embodiment. Rather, inventive aspects lie in a combination of fewer than all features of any single foregoing disclosed embodiment.

Recitation of the term “first” with respect to a feature or element does not necessarily imply the existence of a second or additional such feature or element. Elements recited in means-plus-function format are intended to be construed in accordance with 35 U.S.C. § 112(f). It will be apparent to those having skill in the art that changes may be made to the details of the above-described embodiments without departing from the underlying principles set forth herein.

The phrases “connected to,” “coupled to,” “engaged with,” and “in communication with” refer to any form of interaction between two or more entities, including mechanical, electrical, magnetic, electromagnetic, fluid, and thermal interaction. Two components may be functionally coupled to each other even though they are not in direct contact with each other. The term “coupled” can include components that are coupled to each other via integral formation, as well as components that are removably and/or non-removably coupled with each other. The term “abutting” refers to items that may be in direct physical contact with each other, although the items may not necessarily be attached together. The phrase “fluid communication” refers to two or more features that are connected such that a fluid within one feature is able to pass into another feature. Moreover, as defined herein the term “substantially” means within +/−20% of a target value, measurement, or desired characteristic.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the devices, systems, instruments, and methods disclosed herein.

What is claimed is:

1. A tibial prosthesis system for knee arthroplasty, the tibial prosthesis system comprising:
a tibial tray comprising an anterior portion, a posterior portion, a bone facing surface, a joint facing surface, and a central aperture; and
a clip configured to be slidably received in the central aperture;
wherein:
the posterior portion comprises a posterior notch;
the central aperture extends posteriorly from the anterior portion, and from the bone facing surface to the joint facing surface; and
at least a portion of the joint facing surface extends between the central aperture and the posterior notch.

2. The tibial prosthesis system of claim 1, wherein the central aperture is configured to receive a natural ligament or a ligament graft.

3. The tibial prosthesis system of claim 1, wherein the tibial tray comprises a central axis equidistant between the anterior portion and the posterior portion, and the central aperture extends posteriorly beyond the central axis.

4. The tibial prosthesis system of claim 1, wherein the clip comprises an anterior profile configured to align with an anterior edge of the anterior portion of the tibial tray.

5. The tibial prosthesis system of claim 1, wherein the clip extends from the bone facing surface to the joint facing surface.

6. The tibial prosthesis system of claim 1, wherein, with the clip received in the central aperture, at least a portion of the central aperture remains unobstructed between the bone facing surface and the joint facing surface.

7. The tibial prosthesis system of claim 1, wherein, with the clip received in the central aperture, the central aperture is completely obstructed.

8. The tibial prosthesis system of claim 1, further comprising a first stem and a second stem, wherein both the first stem and the second stem are configured to be embedded within a tibia to inhibit movement of the tibial prosthesis system relative to the tibia.

9. A tibial prosthesis system for knee arthroplasty, the tibial prosthesis system comprising:
a tibial tray comprising an anterior portion, a posterior portion, a central axis, and a central aperture; and
a clip configured to be received in the central aperture;
wherein:
the central axis is equidistant between the anterior portion and the posterior portion;
the posterior portion comprises a posterior notch; and
the central aperture extends posteriorly from the anterior portion beyond the central axis.

10. The tibial prosthesis system of claim 9, wherein the central aperture is configured to receive a natural ligament or a ligament graft.

11. The tibial prosthesis system of claim 9, wherein, with the clip received in the central aperture, at least a portion of the central aperture remains unobstructed.

12. The tibial prosthesis system of claim 9, wherein, with the clip received in the central aperture, the central aperture is completely obstructed.

13. The tibial prosthesis system of claim 9, further comprising a first stem and a second stem, wherein both the first stem and the second stem are configured to be embedded within a tibia to inhibit movement of the tibial prosthesis system relative to the tibia.

14. The tibial prosthesis system of claim 9, wherein the clip comprises an anterior profile configured to align with an anterior edge of the anterior portion of the tibial tray.

15. The tibial prosthesis system of claim 9, wherein the tibial tray further comprises a bone facing surface and a joint facing surface, and the clip extends from the bone facing surface to the joint facing surface.

16. A tibial prosthesis system for knee arthroplasty, the tibial prosthesis system comprising:
a tibial tray comprising an anterior portion, a bone facing surface, a joint facing surface, and a central aperture; and
a clip configured to be received in the central aperture;
wherein:
the central aperture extends posteriorly from the anterior portion, and from the bone facing surface to the joint facing surface; and
the central aperture is configured to receive a natural ligament or a ligament graft.

17. The tibial prosthesis system of claim 16, wherein the tibial tray further comprises a posterior portion and a central axis equidistant between the anterior portion and the posterior portion, and the central aperture extends posteriorly beyond the central axis.

18. The tibial prosthesis system of claim 16, wherein, with the clip received in the central aperture, at least a portion of the central aperture remains unobstructed between the bone facing surface and the joint facing surface.

19. The tibial prosthesis system of claim 16, further comprising a first stem and a second stem, wherein both the first stem and the second stem are configured to be embedded within a tibia to inhibit movement of the tibial prosthesis system relative to the tibia.

20. The tibial prosthesis system of claim 16, wherein the clip comprises an anterior profile configured to align with an anterior edge of the anterior portion of the tibial tray.
